(19) United States
(12) Patent Application Publication
(10) Pub. No.: US 2014/0212431 A1
(43) Pub. Date: Jul. 31, 2014

(54) PCSK9-BINDING POLYPEPTIDES AND METHODS OF USE

(71) Applicant: Genentech, Inc., South San Francisco, CA (US)

(72) Inventors: Daniel K. Kirchhofer, South San Francisco, CA (US); Yingnan Zhang, South San Francisco, CA (US); Andrew Scott Peterson, South San Francisco, CA (US); Wei Li, South San Francisco, CA (US); Monica Kong-Beltran, South San Francisco, CA (US); Lijuan Zhou, South San Francisco, CA (US)

(73) Assignee: GENENTECH, INC., South San Francisco, CA (US)

(21) Appl. No.: 14/133,986

(22) Filed: Dec. 19, 2013

Related U.S. Application Data
(63) Continuation of application No. PCT/US2012/043315, filed on Jun. 20, 2012.
(60) Provisional application No. 61/499,034, filed on Jun. 20, 2011.

(51) Int. Cl. C07K 16/40; A61K 45/06; A61K 39/395

(52) U.S. Cl. CPC C07K 16/40; A61K 39/3955; A61K 45/06; A61K 2039/505
USPC 424/158.1; 435/7.92; 435/69.6; 435/184; 435/338; 435/419; 435/252.3; 435/254.11; 435/254.2; 435/320.1; 436/501; 530/389.1; 536/23.53

(57) ABSTRACT

The invention provides PCSK9-binding polypeptides and methods of using the same.

### RELATED APPLICATIONS

This application is a continuation of International Application No. PCT/US2012/043315 having an international filing date of Jun. 20, 2012, which claims the benefit of U.S. Provisional Application Ser. No. 61/499,034, filed Jun. 20, 2011. All the teachings of the above-referenced applications are incorporated herein by reference.

### SEQUENCE LISTING

The instant application contains a Sequence Listing which has been submitted in ASCII format via EFS-Web and is hereby incorporated by reference in its entirety. Said ASCII copy, created on Dec. 13, 2013, is named P4562C1.txt and is 10,666 bytes in size.

### FIELD OF THE INVENTION

The present invention relates to polypeptides that bind to PCSK9 and methods of using the same.

### BACKGROUND OF THE INVENTION

Proprotein convertase subtilisin/kexin type 9 (PCSK9) is a member of the mammalian subtilisin family of proprotein convertases and functions as a strong negative regulator of hepatic LDL receptors (LDLR). PCSK9 plays a critical role in cholesterol metabolism by controlling the levels of low density lipoprotein (LDL) particles that circulate in the bloodstream. Elevated levels of PCSK9 have been shown to reduce LDL-receptor levels in the liver, resulting in high levels of LDL-cholesterol in the plasma and increased susceptibility to coronary artery disease. (Peterson et al., J Lipid Res. 49(7):1595-9 (2008)). Therefore, it would be highly advantageous to produce a therapeutic-based antagonist of PCSK9 that inhibits or antagonizes the activity of PCSK9 and the corresponding role PCSK9 plays in various pathologic conditions.

### SUMMARY OF THE INVENTION

The invention is in part based on a variety of polypeptides that bind to PCSK9. PCSK9 presents as an important and advantageous therapeutic target, and the invention provides PCSK9-binding polypeptides as therapeutic and diagnostic agents for use in targeting pathological conditions associated with expression and/or activity of PCSK9. Accordingly, the invention provides methods, compositions, kits and articles of manufacture related to PCSK9.

In one aspect, the invention provides a PCSK9-binding polypeptide comprising the amino acid sequence: GX₁X₂ECLX₃NX₄GGCSX₅X₆CX₇X₈LKIGYECLCPDGFQLVAQRRCE, wherein X₁ is D or T; X₂ is L or N; X₃ is selected from the group consisting of A, D, E, H, K, L, R, S, V, and Y; X₄ is L or N; X₅ is selected from the group consisting of H, W, and Y; X₆ is selected from the group consisting of I, L, T and V; X₇ is selected from the group consisting of K, N, R and Q; and X₈ is selected from the group consisting of A, D, K, N, Q and R (SEQ ID NO: 1). In some embodiment, the polypeptide comprises an an amino acid sequence selected from the group consisting of SEQ ID NOs: 2-27 (e.g. the non-wild-type sequences shown in FIG. 2). In some embodiments, the polypeptide further comprises an immunoglobulin sequence, e.g. an antibody constant region (e.g. an Fc region), which may be, e.g., from an IgG antibody.

In some embodiments, the invention provides an isolated nucleic acid encoding a polypeptide of the invention. In some embodiments, the invention provides a vector comprising a nucleic acid encoding such a polypeptide, e.g. an expression vector. In some embodiments, the invention provides a host cell comprising such a vector. Such a host cell can be, e.g. a prokaryotic or eukaryotic host cell.

In some embodiments, the invention provides a method for making the polypeptide of the invention comprising culturing a host cell containing a nucleic acid or vector of the invention under conditions suitable for expression. In some embodiments, the method further comprises recovering the polypeptide from the host cell.

In some embodiments, the invention provides a pharmaceutical composition comprising a polypeptide of the invention and a pharmaceutically acceptable carrier.

In some embodiments, the invention provides a method of reducing LDL-cholesterol level in a subject, said method comprising administering to the subject an effective amount of the polypeptide of the invention. In some embodiments, the invention provides a method of treating cholesterol related disorder in a subject, said method comprising administering to the subject an effective amount of the polypeptide of the invention. In some embodiments, the invention provides a method of treating hypercholesterolemia in a subject, said method comprising administering to the subject an effective amount of the polypeptide of the invention. In some embodiments, these methods further comprise administering to the subject an effective amount of a second medicament, wherein the polypeptide is the first medicament. In some embodiments, the second medicament elevates the level of LDLR. In some embodiments, the second medicament reduces the level of LDL-cholesterol. In some embodiments, the second medicament comprises a statin. In some embodiments, the statin is selected from the group consisting of atorvastatin, fluvastatin, lovastatin, mevastatin, pitavastatin, pravastatin, rosuvastatin, simvastatin, and any combination thereof. In some embodiments, the second medicament elevates the level of HDL-cholesterol.

In some embodiments, the invention provides a method of inhibiting binding of PCSK9 to LDLR in a sample comprising adding a polypeptide of the invention to the sample. In some embodiments, the invention provides a method of inhibiting binding of PCSK9 to LDLR in a subject comprising administering to the subject an effective amount of a polypeptide of the invention.

In some embodiments, the invention provides method of detecting PCSK9 protein in a sample comprising contacting the sample with a polypeptide of the invention and detecting formation of a complex between the polypeptide and the PCSK9 protein.

Any embodiment described herein or any combination thereof applies to any and all PCSK9-binding polypeptides, methods and uses of the invention described herein.

### BRIEF DESCRIPTION OF THE FIGURES

FIG. 1 shows a portion of crystal structure of PCSK9 bound to LDLR and highlights certain residues on the EGF(A) domain of LDLR that are within 3.5 Å of PCSK9.

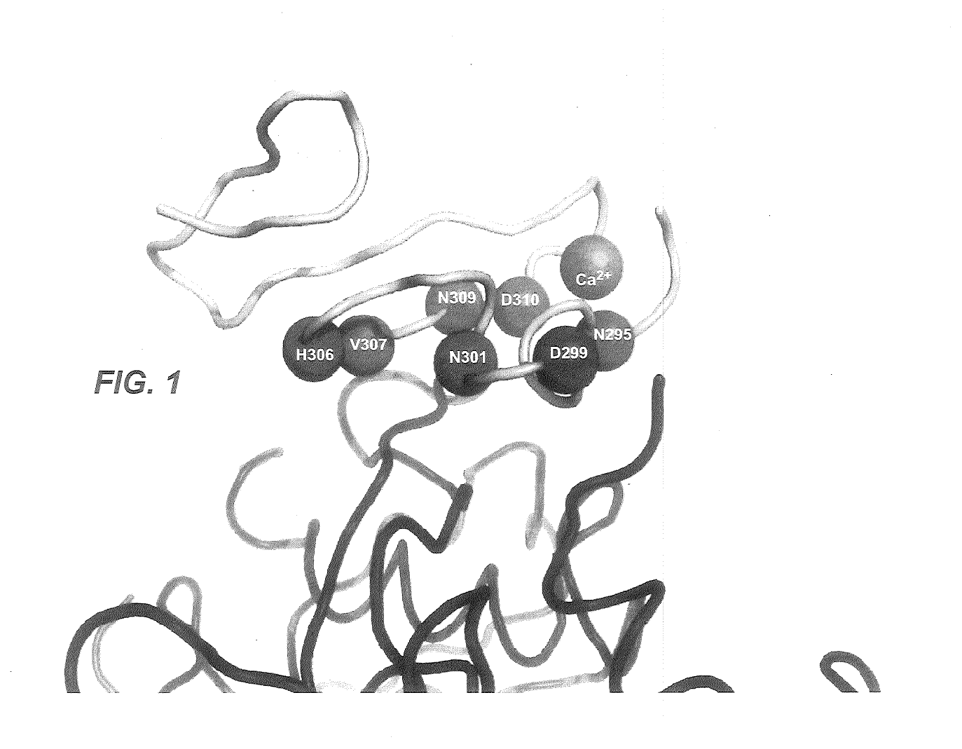

FIG. 2 shows sequences of the variable region (293-312) of wild-type EGF as the first sequence (SEQ ID NO: 28) and variants selected from the EGF library (SEQ ID NOs: 2-27, respectively). The constant region (313-332), with sequence of IGYECLCPDGFQLVAQRRCE (SEQ ID NO: 29), is the same for all clones and not shown. The position numbering are those from the full length LDLR. “s/n ratio” refers to signal:noise ratio, wherein “signal” is the spot phage ELISA signal detected against biotinylated PCSK9 captured by NeutrAvidin coated on the 384-well MaxiSorp™ plate; “noise” is the ELISA signal against NeutrAvidin alone.

FIG. 3 shows the inhibitory activities of EGF peptides (A) and EGF-Fc fusion proteins (B) as determined by a competition binding ELISA. Serial dilutions of competitors were mixed with 0.5 μM biotinylated PCSK9 and added to plates coated with rLDLR. Bound biotinylated PCSK9 was detected by Streptavidin-HRP. Values are the average ±SD of three independent experiments.

FIG. 4 shows EGFwt-Fc or EGF66-Fc were captured by the sensor chip coated with anti-human Fc. Sensorgrams for EGFwt-Fc (A) or EGF66-Fc (B) were recorded by injecting PCSK9 solution ranging from 0.078-10 μM for EGFwt-Fc or 0-2.5 μM for EGF66-Fc in the presence of 1 mM CaCl₂ (upper panel) or 10 mM EDTA (lower panel).

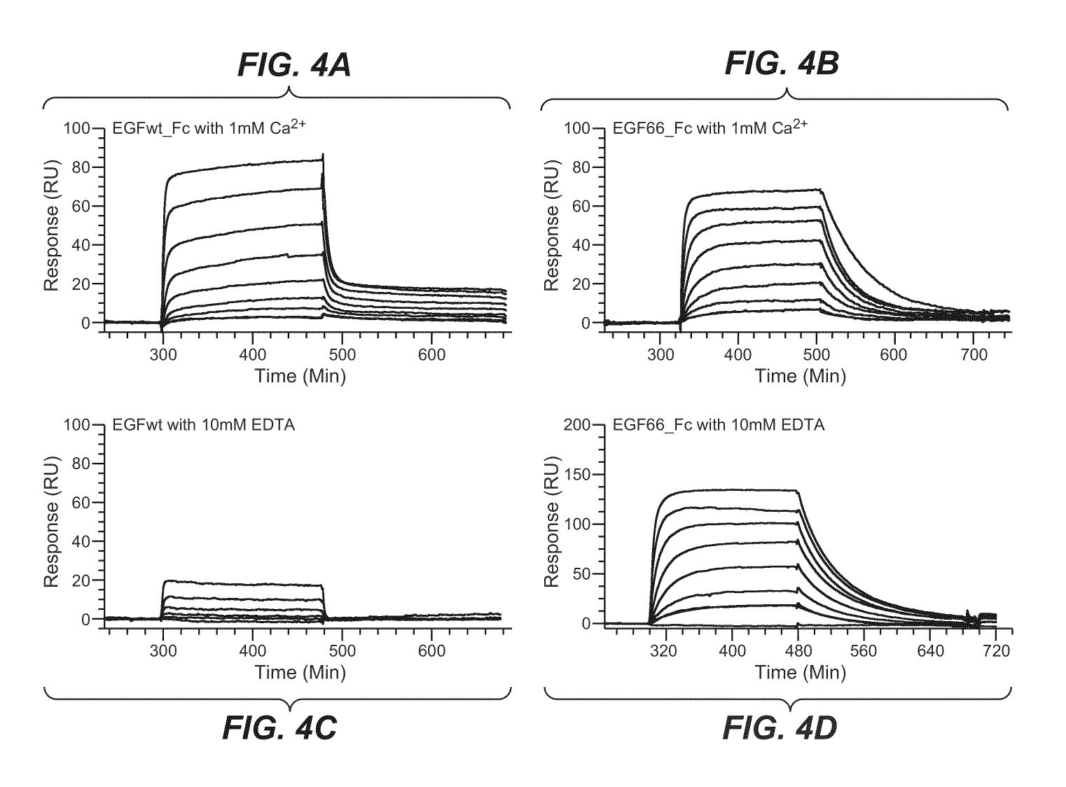

FIG. 5 shows LDLR levels on the HepG2 cell surface monitored by FACS upon treatment of PCSK9 in the presence of EGFwt-Fc or EGF66-Fc. Relative fluorescence units (RFUs) were used to quantify LDLR expression levels and were expressed as percentage of control cells that did not receive PCSK9. Values are the average ±SD of three independent experiments.

FIG. 6 shows the ability of EGFwt-Fc and EGF66-Fc to rescue liver LDLR level upon treatment of PCSK9 in a mouse model. Mice were injected with vehicle (V), EGF-Fc (WT) or EGF66-Fc (MUT) followed by a bolus injection of recombinant human PCSK9 (30 μg/mouse). Livers were collected after 1 h and LDLR quantified by immuno-blotting. Each lane represents the pooled liver samples of three mice. The band intensities were quantified, normalized to transferring receptor contents and expressed as fraction of the untreated group (=1.0).

FIG. 7 shows that the D310K mutation abolishes binding of phage-displayed EGF to PCSK9. The binding curve was measured by phage ELISA in which the EGF-displaying phage with 1:3 serial dilution were added to plate-immobilized PCSK9 and the bound phage were detected by anti-M13-HRP.

FIG. 8 shows SEC-MALS analysis of EGF66-Fc/PCSK9 complex. The Size exclusion chromatography (SEC) profile of EGF66-Fc and PCSK9 injected alone are shown as blue and red traces. The EGF66:PCSK9 mixture with 1:3 or 3:1 molar ratios were injected and SEC profiles were shown as green and black. The average molecular mass (kDa), determined by multi-angle light scattering (MALS), is indicated for each peak. The molecular mass of the first peak is consistent with a stoichiometry of 1:2 (1 EGF66-Fc and 2 PCSK9), and the second peak with 1:1.

FIG. 9 shows Molecular modeling of EGF66. (A) Modeled changes for the D299A, N301L, V307I, N309R and D310K mutations in EGF66 indicating the potential for improved contacts with PCSK9. The backbone of the EGF domain is show as a ribbon with the modeled, mutated residues shown as sticks. N295 and H306 remained as wild-type during the selection and are shown as sticks. Potential lipophilic interactions with the mutated residues are shown with lighter shading and italicized labels on the otherwise grey surface of PCSK9. The surface adjacent to the catalytic triad residues of PCSK9 are shaded a darker grey, and S153 (N-terminus created by autolytic processing of PCSK9) is labeled with an “N”. (B) Model of the D310K side chain in EGF66 in which the terminal amine replaces the need for a Ca²⁺ ion to stabilize the packing of the N-terminal strand onto the β-hairpin. Dotted lines in the left panel indicate atoms within 3.0 Å of the Ca²⁺. Dotted lines in the right panel indicate potential hydrogen bond interactions between the lysine side chain and atoms in the Ca²⁺-binding loop. Note that the actual atoms forming hydrogen bonds will depend on the exact location of the terminal ammonium group of the lysine.

### DETAILED DESCRIPTION OF EMBODIMENTS OF THE INVENTION

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3rd. edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993).

### I. DEFINITIONS

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application. All references cited herein, including patent applications and publications, are incorporated by reference in their entirety.

For purposes of interpreting this specification, the following definitions will apply and whenever appropriate, terms used in the singular will also include the plural and vice versa. It is to be understood that the terminology used herein is for the purpose of describing particular embodiments only, and is not intended to be limiting. In the event that any definition set forth below conflicts with any document incorporated herein by reference, the definition set forth below shall control.

“Affinity” refers to the strength of the sum total of noncovalent interactions between a single binding site of a molecule (e.g., an polypeptide) and its binding partner (e.g., another polypeptide). Unless indicated otherwise, as used herein, “binding affinity” refers to intrinsic binding affinity which reflects a 1:1 interaction between members of a binding pair (e.g., ligand and receptor). The affinity of a molecule X for its partner Y can generally be represented by the dissociation constant (“Kd” or “KD”). Affinity can be measured by common methods known in the art, including those described herein. Specific illustrative and exemplary embodiments for measuring binding affinity are described in the following.

The terms “PCSK9-binding polypeptide” or “polypeptide that binds to PCSK9” refers to a polypeptide that is capable of binding PCSK9 with sufficient affinity such that the polpeptide is useful as a diagnostic and/or therapeutic agent in targeting PCSK9. In one embodiment, the extent of binding of a PCSK9-binding polypeptide to an unrelated, non-PCSK9 protein is less than about 10% of the binding of the binding to PCSK9 as measured, e.g., by quantitative ELISA.

An “effective amount” of an agent, e.g., a pharmaceutical formulation, refers to an amount effective, at dosages and for periods of time necessary, to achieve the desired therapeutic or prophylactic result.

The term “Fc region” herein is used to define a C-terminal region of an immunoglobulin heavy chain that contains at least a portion of the constant region. The term includes native sequence Fc regions and variant Fc regions. In certain embodiments, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The term “hypercholesterolemia,” as used herein, refers to a condition in which cholesterol levels are elevated above a desired level. In certain embodiments, the LDL-cholesterol level is elevated above the desired level. In certain embodiments, the serum LDL-cholesterol levels are elevated above the desired level.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “isolated” polypeptide is one which has been separated from a component of its natural environment. In some embodiments, a polypeptide is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “pharmaceutical formulation” or “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “proprotein convertase subtilisin kexin type 9,” “PCSK9,” or “NARC-1,” as used herein, refers to any native PCSK9 from any vertebrate source, including mammals such as primates (e.g. humans) and rodents (e.g., mice and rats), unless otherwise indicated. The term encompasses “full-length,” unprocessed PCSK9 as well as any form of PCSK9 that results from processing in the cell or any fragment thereof. The term also encompasses naturally occurring variants of PCSK9, e.g., splice variants or allelic variants.

The term “PCSK9 activity” or “biological activity” of PCSK9, as used herein, includes any biological effect of PCSK9. In certain embodiments, the biological activity of PCSK9 is the ability of PCSK9 to bind to a LDL-receptor (LDLR). In certain embodiments, PCSK9 binds to and catalyzes a reaction involving LDLR. In certain embodiments, PCSK9 activity includes the ability of PCSK9 to decrease or reduce the availability of LDLR. In certain embodiments, the biological activity of PCSK9 includes the ability of PCSK9 to increase the amount of LDL in a subject. In certain embodiments, the biological activity of PCSK9 includes the ability of PCSK9 to decrease the amount of LDLR that is available to bind to LDL in a subject. In certain embodiments, the biological activity of PCSK9 includes the ability of PCSK9 to decrease the amount of LDLR that is available to bind to LDL. In certain embodiments, biological activity of PCSK9 includes any biological activity resulting from PCSK9 signaling.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### II. COMPOSITIONS AND METHODS

In one aspect, the invention is based, in part, on experimental results obtained with PCSK9-binding polypeptides. Results obtained indicate that blocking biological activity of PCSK9 with these polypeptides leads to a prevention of reduction in LDLR. Accordingly, PCSK9-binding polypeptides of the invention, as described herein, provide important therapeutic and diagnostic agents for use in targeting pathological conditions associated with PCSK9, e.g., cholesterol related disorders.

In certain embodiments, a “cholesterol related disorder” includes any one or more of the following: hypercholesterolemia, heart disease, metabolic syndrome, diabetes, coronary heart disease, stroke, cardiovascular diseases, Alzheimers disease and generally dyslipidemias, which can be manifested, for example, by an elevated total serum cholesterol, elevated LDL, elevated triglycerides, elevated VLDL, and/or low HDL. Some non-limiting examples of primary and secondary dyslipidemias that can be treated using a PCSK9-binding polypeptide, either alone, or in combination with one or more other agents include the metabolic syndrome, diabetes mellitus, familial combined hyperlipidemia, familial hypertriglyceridemia, familial hypercholesterolemias, including heterozygous hypercholesterolemia, homozygous hypercholesterolemia, familial defective apoplipoprotein B-100; polygenic hypercholesterolemia; remnant removal disease, hepatic lipase deficiency; dyslipidemia secondary to any of the following: dietary indiscretion, hypothyroidism, drugs including estrogen and progestin therapy, beta-blockers, and thiazide diuretics; nephrotic syndrome, chronic renal failure, Cushing's syndrome, primary biliary cirrhosis, glycogen storage diseases, hepatoma, cholestasis, acromegaly, insulinoma, isolated growth hormone deficiency, and alcohol-induced hypertriglyceridemia. PCSK9-binding polypeptides described herein can also be useful in preventing or treating atherosclerotic diseases, such as, for example, coronary heart disease, coronary artery disease, peripheral arterial disease, stroke (ischaemic and hemorrhagic), angina pectoris, or cerebrovascular disease and acute coronary syndrome, myocardial infarction. In certain embodiments, the PCSK9-binding polypeptides described herein are useful in reducing the risk of: nonfatal heart attacks, fatal and non-fatal strokes, certain types of heart surgery, hospitalization for heart failure, chest pain in patients with heart disease, and/or cardiovascular events because of established heart disease such as prior heart attack, prior heart surgery, and/or chest pain with evidence of clogged arteries. In certain embodiments, the PCSK9-binding polypeptides and methods described herein can be used to reduce the risk of recurrent cardiovascular events.

A. Recombinant Methods and Compositions

PCSK9-binding polypeptides described herein may be produced using recombinant methods and compositions, e.g., as described in U.S. Pat. No. 4,816,567. In one embodiment, isolated nucleic acid encoding a PCSK9-binding polypeptide described herein is provided. In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with) a vector comprising a nucleic acid that encodes a PCSK9-binding polypeptide. In one embodiment, the host cell is eukaryotic, e.g. a Chinese Hamster Ovary (CHO) cell or lymphoid cell (e.g., Y0, NS0, Sp20 cell). In one embodiment, a method of making a PCSK9-binding polypeptide is provided, wherein the method comprises culturing a host cell comprising a nucleic acid encoding the polypeptide, as provided above, under conditions suitable for expression of the polypeptide, and optionally recovering it from the host cell (or host cell culture medium).

For recombinant production of a PCSK9-binding polypeptide, nucleic acid encoding a PCSK9-binding polypeptide, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of PCSK9-binding polypeptide-encoding vectors include prokaryotic or eukaryotic cells described herein. For example, PCSK9-binding polypeptide may be produced in bacteria, in particular when glycosylation is not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B.K.C. Lo, ed., Humana Press, Totowa, N.J., 2003), pp. 245-254, describing expression of antibody fragments in E. coli.). After expression, the PCSK9-binding polypeptide may be isolated from the bacterial cell paste in a soluble fraction and can be further purified.

In addition to prokaryotes, eukaryotic microbes such as filamentous fungi or yeast are suitable cloning or expression hosts for PCSK9-binding polypeptide-encoding vectors, including fungi and yeast strains whose glycosylation pathways have been “humanized,” resulting in the production of a PCSK9-binding polypeptide with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated PCSK9-binding polypeptide are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0.

B. Assays

PCSK9-binding polypeptides provided herein may be identified, screened for, or characterized for their physical/chemical properties and/or biological activities by various assays known in the art.

1. Binding Assays and Other Assays

In one aspect, a PCSK9-binding polypeptide of the invention is tested for its PCSK9 binding activity, e.g., by known methods such as ELISA, Western blot, etc. In some embodiments, a PCSK9-binding polypeptide of the invention is tested for its PCSK9 binding activity by bio-layer interferometry or surface plasmon resonance.

2. Activity Assays

In one aspect, assays are provided for identifying PCSK9-binding polypeptides thereof having biological activity. Biological activity of the PCSK9-binding polypeptides may include, e.g., blocking, antagonizing, suppressing, interfering, modulating and/or reducing one or more biological activities of PCSK9. PCSK9-binding polypeptides having such biological activity in vivo and/or in vitro are provided.

In certain embodiments, PCSK9-binding polypeptide binds human PCSK9 and prevents interaction with the LDLR. In certain embodiments, PCSK9-binding polypeptide binds specifically to human PCSK9 and/or substantially inhibits binding of human PCSK9 to LDLR by at least about 20%-40%, 40-60%, 60-80%, 80-85%, or more (for example, by measuring binding in an in vitro competitive binding assay). In certain embodiments, the invention provides isolated PCSK9-binding polypeptides which specifically bind to PCSK9 and which antagonize the PCSK9-mediated effect on LDLR levels when measured in vitro using the LDLR down regulation assay in HepG2 cells disclosed herein.

C. Methods and Compositions for Diagnostics and Detection

In certain embodiments, any of the PCSK9-binding polypeptides provided herein is useful for detecting the presence of PCSK9 in a biological sample. The term “detecting” as used herein encompasses quantitative or qualitative detection. In certain embodiments, a biological sample is blood, serum or other liquid samples of biological origin. In certain embodiments, a biological sample comprises a cell or tissue.

In one embodiment, a PCSK9-binding polypeptide for use in a method of diagnosis or detection is provided. In a further aspect, a method of detecting the presence of PCSK9 in a biological sample is provided. In certain embodiments, the method comprises detecting the presence of PCSK9 protein in a biological sample. In certain embodiments, PCSK9 is human PCSK9. In certain embodiments, the method comprises contacting the biological sample with a PCSK9-binding polypeptide as described herein under conditions permissive for binding of the PCSK9-binding polypeptide to PCSK9, and detecting whether a complex is formed between the PCSK9-binding polypeptide and PCSK9. Such method may be an in vitro or in vivo method. In one embodiment, a PCSK9-binding polypeptide is used to select subjects eligible for therapy with a PCSK9-binding polypeptide, e.g. where PCSK9 or LDL-cholesterol is a biomarker for selection of patients.

Exemplary disorders that may be diagnosed using a polypeptide of the invention include cholesterol related disorders (which includes “serum cholesterol related disorders”), including any one or more of the following: hypercholesterolemia, heart disease, metabolic syndrome, diabetes, coronary heart disease, stroke, cardiovascular diseases, Alzheimers disease and generally dyslipidemias, which can be manifested, for example, by an elevated total serum cholesterol, elevated LDL, elevated triglycerides, elevated very low density lipoprotein (VLDL), and/or low HDL. In one aspect, the invention provides a method for treating or preventing hypercholesterolemia, and/or at least one symptom of dyslipidemia, atherosclerosis, cardiovascular disease (CVD) or coronary heart disease, in an individual comprising administering to the individual an effective amount of PCSK9-binding polypeptide. In certain embodiments, the invention provides an effective amount of a PCSK9-binding polypeptide for use in treating or preventing hypercholesterolemia, and/or at least one symptom of dyslipidemia, atherosclerosis, CVD or coronary heart disease, in a subject. The invention further provides the use of an effective amount of a PCSK9-binding polypeptide that antagonizes extracellular or circulating PCSK9 in the manufacture of a medicament for treating or preventing hypercholesterolemia, and/or at least one symptom of dyslipidemia, atherosclerosis, CVD or coronary heart disease, in an individual.

In certain embodiments, labeled PCSK9-binding polypeptides are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

D. Pharmaceutical Formulations

This invention also encompasses compositions, including pharmaceutical formulations, comprising a PCSK9-binding polypeptide, and polynucleotides comprising sequences encoding a PCSK9-binding polypeptide. In certain embodiments, compositions comprise one or more polypeptides that bind to PCSK9, or one or more polynucleotides comprising sequences encoding one or more polypeptides that bind to PCSK9. These compositions may further comprise suitable carriers, such as pharmaceutically acceptable excipients including buffers, which are well known in the art.

Pharmaceutical formulations of a PCSK9-binding polypeptide as described herein are prepared by mixing such polypeptide having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

The formulation herein may also contain more than one active ingredients as necessary for the particular indication being treated, preferably those with complementary activities that do not adversely affect each other. For example, it may be desirable to further provide statin. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

E. Therapeutic Methods and Compositions

Any of the PCSK9-binding polypeptides provided herein may be used in therapeutic methods.

In one aspect, a PCSK9-binding polypeptide for use as a medicament is provided. In another aspect, a PCSK9-binding polypeptide for use in treating conditions associated with cholesterol related disorder is provided. In certain embodiments, a PCSK9-binding polypeptide for use in treating conditions associated with elevated level of LDL-cholesterol is provided. In certain embodiments, a PCSK9-binding polypeptide for use in a method of treatment is provided. In certain embodiments, the invention provides a PCSK9-binding polypeptide for use in a method of treating an individual having conditions associated with elevated level of LDL-cholesterol comprising administering to the individual an effective amount of the PCSK9-binding polypeptide. In certain embodiments, the methods and uses described herein further comprise administering to the individual an effective amount of at least one additional therapeutic agent, e.g., statin. In certain embodiments, the invention provides a PCSK9-binding polypeptide for use in reducing LDL-cholesterol level in a subject. In further embodiments, the invention provides a PCSK9-binding polypeptide for use in lowering serum LDL-cholesterol level in a subject. In certain embodiments, the invention provides a PCSK9-binding polypeptide for use in increasing availability of LDLR in a subject. In certain embodiments, the invention provides a PCSK9-binding polypeptide for use in inhibiting binding of PCSK9 to LDLR in a subject. In certain embodiments, the invention provides a PCSK9-binding polypeptide for use in a method of reducing LDL-cholesterol level in an individual comprising administering to the individual an effective of the PCSK9-binding polypeptide to reduce the LDL-cholesterol level. In certain embodiments, the invention provides a PCSK9-binding polypeptide for use in a method of lowering serum LDL-cholesterol level in an individual comprising administering to the individual an effective of the PCSK9-binding polypeptide to lower the serum LDL-cholesterol level. In certain embodiments, the invention provides a PCSK9-binding polypeptide for use in a method of increasing availability of LDLR in an individual comprising administering to the individual an effective of the PCSK9-binding polypeptide to increase availability of LDLR. In certain embodiments, the invention provides a PCSK9-binding polypeptide for use in a method of inhibiting binding of PCSK9 to LDLR in an individual comprising administering to the individual an effective amount of the PCSK9-binding polypeptide to inhibit the binding of PCSK9 to LDLR. An “individual” according to any of the above embodiments is preferably a human.

In a further aspect, the invention provides for the use of a PCSK9-binding polypeptide in the manufacture or preparation of a medicament. In one embodiment, the medicament is for treatment of cholesterol related disorder. In certain embodiments, the cholesterol related disorder is hypercholesterolemia. In another embodiment, the medicament is for use in a method of treating hypercholesterolemia comprising administering to an individual having hypercholesterolemia an effective amount of the medicament.

In certain embodiments, the disorder treated is any disease or condition which is improved, ameliorated, inhibited or prevented by removal, inhibition or reduction of PCSK9 activity. In certain embodiments, diseases or disorders that are generally addressable (either treatable or preventable) through the use of statins can also be treated. In certain embodiments, disorders or disease that can benefit from the prevention of cholesterol synthesis or increased LDLR expression can also be treated by PCSK9-binding polypeptide of the present invention. In certain embodiments, individuals treatable by the PCSK9-binding polypeptides and therapeutic methods of the invention include individuals indicated for LDL apheresis, individuals with PCSK9-activating mutations (gain of function mutations, “GOF”), individuals with heterozygous Familial Hypercholesterolemia (heFH), individuals with primary hypercholesterolemia who are statin intolerant or statin uncontrolled, and individuals at risk for developing hypercholesterolemia who may be presentably treated. Other indications include dyslipidemia associated with secondary causes such as Type 2 diabetes mellitus, cholestatic liver diseases (primary biliary cirrhosis), nephrotic syndrome, hypothyroidism, obesity, and the prevention and treatment of atherosclerosis and cardiovascular diseases.

In certain embodiments, the methods and uses described herein further comprises administering to the individual an effective amount of at least one additional therapeutic agent, e.g., statin. In certain embodiments, the additional therapeutic agent is for preventing and/or treating atherosclerosis and/or cardiovascular diseases. In certain embodiment, the additional therapeutic agent is for use in a method of reducing the risk of recurrent cardiovascular events. In certain embodiments, the additional therapeutic agent is for elevating the level of HDL-cholesterol in a subject.

In a further aspect, the invention provides pharmaceutical formulations comprising any of the PCSK9-binding polypeptides provided herein, e.g., for use in any of the above therapeutic methods. In one embodiment, a pharmaceutical formulation comprises any of the PCSK9-binding polypeptides provided herein and a pharmaceutically acceptable carrier. In another embodiment, a pharmaceutical formulation comprises any of the PCSK9-binding polypeptides provided herein and at least one additional therapeutic agent, e.g., statin.

PCSK9-binding polypeptide of the invention can be used either alone or in combination with other agents in a therapy. For instance, a PCSK9-binding polypeptide of the invention may be co-administered with at least one additional therapeutic agent. In certain embodiments, such additional therapeutic agent elevates the level of LDLR. In certain embodiments, an additional therapeutic agent is a LDL-cholesterol lowering drugs such as statin, e.g., atorvastatin, fluvastatin, lovastatin, mevastatin, pitavastatin, pravastatin, rosuvastatin, simvastatin, or any combination thereof, e.g., VYTORIN®, ADVICOR® or SIMCOR®. In certain embodiments, an additional therapeutic agent is a HDL-cholesterol raising drugs.

Such combination therapies noted above encompass combined administration (where two or more therapeutic agents are included in the same or separate formulations), and separate administration, in which case, administration of the PCSK9-binding polypeptide of the invention can occur prior to, simultaneously, and/or following, administration of the additional therapeutic agent and/or adjuvant.

A PCSK9-binding polypeptide of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

PCSK9-binding polypeptides of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The PCSK9-binding polypeptide need not be, but is optionally formulated with one or more agents currently used to prevent or treat the disorder in question. The effective amount of such other agents depends on the amount of PCSK9-binding polypeptide present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of a PCSK9-binding polypeptide of the invention (when used alone or in combination with one or more other additional therapeutic agents) will depend on the type of disease to be treated, the severity and course of the disease, whether the polypeptide is administered for preventive or therapeutic purposes, previous therapy, the patient's clinical history and response to the polypeptide, and the discretion of the attending physician. The PCSK9-binding polypeptide is suitably administered to the patient at one time or over a series of treatments. Depending on the type and severity of the disease, about 1 μg/kg to 15 mg/kg (e.g. 0.1 mg/kg-10 mg/kg) of PCSK9-binding polypeptide can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the PCSK9-binding polypeptide would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the polypeptide). An initial higher loading dose, followed by one or more lower doses may be administered.

In certain embodiments, a flat-fixed dosing regimen is used to administer PCSK9-binding polypeptide to an individual. Depending on the type and severity of the disease an exemplary flat-fixed dosage might range from 10 to 1000 mg of PCSK9-binding polypeptide. One exemplary dosage of the polypeptide would be in the range from about 10 mg to about 600 mg. Another exemplary dosage of the polypeptide would be in the range from about 100 mg to about 600 mg. In certain embodiments, 150 mg, 300 mg, or 600 mg of PCSK9-binding polypeptide is administered to an individual. However, other dosage regimens may be useful. The progress of this therapy is easily monitored by conventional techniques and assays.

F. Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a PCSK9-binding polypeptide of the invention. The label or package insert indicates that the composition is used for treating the condition of choice. Moreover, the article of manufacture may comprise (a) a first container with a composition contained therein, wherein the composition comprises a PCSK9-binding polypeptide of the invention; and (b) a second container with a composition contained therein, wherein the composition comprises a further cytotoxic or otherwise therapeutic agent. In certain embodiments, the second container comprises a second therapeutic agent, wherein the second therapeutic agent is a cholesterol-lowering drug of the “statin” class. In certain embodiments, the statin is and/or comprises atorvastatin (e.g., LIPITOR® or Torvast), fluvastatin (e.g., LESCOL®), lovastatin (e.g., MEVACOR®, ALTOCOR™, or ALTOPREV®), mevastatin (pitavastatin (e.g., LIVALO® or PITAVA®), pravastatin (e.g., PRAVACHOL®, SELEKTINE®, LIPOSTAT®), rosuvastatin (e.g., CRESTOR®), simvastatin (e.g., ZOCOR®, LIPEX®), or any combination thereof, e.g., VYTORIN®, ADVICOR® or SIMCOR®.

The article of manufacture in this embodiment of the invention may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### III. EXAMPLES

The following are examples of methods and compositions of the invention. It is understood that various other embodiments may be practiced, given the general description provided above.

### Example 1

### Generation of High Affinity PCSK9-Binding Polypeptides

PCSK9 binds to the first epidermal growth factor-like domain, EGF(A), of LDLR and structural studies revealed that the EGF(A) binding site is located on the protease domain (Kwon, et al. (2008) Proc Natl Acad Sci USA 105(6), 1820-1825). A naturally occurring PCSK9 gain-of-function mutation D374Y (Cunningham, et al. (2007) Nat Struct Mol Biol 14(5), 413-419; Lagace, et al. (2006) J Clin Invest 116(11), 2995-3005; Timms, et al. (2004) Hum Genet. 114(4), 349-353) is located at the periphery of the PCSK9-EGF(A) interface region and is in proximity to the familial hypercholesterolemia-associated mutation H306Y in the EGF(A) domain. The structure of the complex also provided a molecular basis to understand the observed affinity increases of the PCSK9-D374Y and EGF-H306Y mutations (Kwon, et al., supra).

The wild-type LDLR-EGF(A) domain alone and the EGF(A,B) tandem domain are competitive inhibitors of LDLR binding to PCSK9 and can partially restore LDLR levels in cell-based assays (Shan, et al. (2008) Biochem Biophys Res Commun 375(1), 69-73; Bottomley, et al. (2009) J Biol Chem 284(2), 1313-1323; McNutt, et al. (2009) J Biol Chem 284(16), 10561-10570). However, the binding affinity of wild-type EGF(A) to PCSK9 is low, with a reported KD value of ˜1 μM at neutral pH (Shan, et al., supra), while the affinity of EGF(A,B) is only slightly better (KD 0.34 μM) (Bottomley, et al., supra). Therefore, the wild-type EGF(A) domain lacks adequate potency for consideration as a potential PCSK9 neutralizing agent.

To identify more potent EGF(A) domain inhibitors, we designed an EGF(A) library with a theoretical diversity of 10⁹ for surface display on phage and identified multiple EGF variants with improved binding affinities and antagonistic activities were identified. The EGF(A) domain of LDLR (G293-E332) was displayed on the surface of M13 bacteriophage by modifying a previously described phagemid pS2202d (Skelton, et al. (2003) J Biol Chem 278(9), 7645-7654). Standard molecular biology techniques were used to replace the fragment of pS2202d encoding gD tag and Erbin PDZ domain with a DNA fragment encoding for EGF(A) domain of LDLR. The resulting phagemid (p3EGF(A)) contained an open reading frame that encoded for the maltose binding protein secretion signal, followed by EGF(A) and ending with the C-terminal domain of M13 minor coat protein p3. E. coli harboring p3EGF(A) were co-infected with M13-KO7 helper phage and cultures were grown in 30 ml 2YT medium supplemented with 50 μg/ml carbenecillin and 25 μg/ml kanamycin at 30° C. for overnight. The propagated phage was purified according to a standard protocol (Tonikian, et al. (2007) Nat Protoc 2(6), 1368-1386) and re-suspended in 1 ml PBT buffer (PBS, 0.5% BSA and 0.1% Tween®20), resulting in the production of phage particles that encapsulated p3EGF(A) DNA and displayed EGF(A) domain. The display level was analyzed using a phage ELISA.

The library was designed by randomizing EGF(A) residues that were within 3.5 Å distance from PCSK9 (exclusind cysteines) based on the crystal structure of the PCSK9:EGF(A,B) complex (Kwon, et al., supra). In order to maximize the library diversity, residues of the Ca²⁺-binding loops (N-terminal and β-hairpin loops) were also randomized and no attempt was made to preserve Ca²⁺-binding, carrying out phage panning in Ca²⁺-free buffer. The EGF(A) domain mutation libraries were constructed following the Kunkel mutagenesis method (Kunkel, et al. (1987) Methods Enzymol 154, 367-382). Residues N295, D299, N301, H306, V307, N309 and D310 were randomized with the NNK codon. The stop template is the single strand DNA of p3EGF(A) containing three stop codons in the H306-D310 region and was used to construct the library that contained ˜2×10¹⁰ unique members. The library was cycled through rounds of binding selection in solution against biotinylated PCSK9. For round one, 20 μg of biotinylated PCSK9 was incubated with 1 ml of phage library (˜1×10¹³ pfu/ml) at 4° C. for 2 h in PBS, 1% BSA and 0.1% Tween20 and captured for 15 min at room temperature by 200 μl of Dynabeads® MyOne Streptavidin that has been previously blocked with blocking buffer (PBS, 1% BSA). The supernatant was discarded and the beads were washed three times with PBS, 0.1% Tween®20. The bound phage was eluted with 400 μl 0.1 M HCl for 7 min and immediately neutralized with 60 μl of 1 M Tris, pH 13. The eluted phage was amplified as described by Tonikian et al. (2007) Nat Protoc 2(6), 1368-1386. For round two, the protocol was the same as round one except for using 10 μg biotinylated PCSK9 and 100 μl of Dynabeads. For round three, 2 μg biotinylated PCSK9 was incubated with the amplified phage from the previous round and the phage-PCSK9 complex was captured by NeutrAvidin-coated plates previously treated with blocking buffer. Round four was identical to round three except for using Strepavidin-coated plates to capture biotin-PCSK9-phage complex. Phage was propagated in E. coli XL1-blue with M13-KO7 helper phage at 30° C.

After four rounds of binding selection, individual phage clones were picked and inoculated into 450 μl 2YT media containing 50 μg/ml carbenecillin and M13-KO7 helper phage in 96-well blocks, which were grown at 37° C. for overnight. The supernatant was analyzed with spot phage ELISA as follows: Biotinylated PCSK9 was captured to NeutrAvidin-coated 384-well MaxiSorp™ immunoplates and phage supernatant diluted (1:3) with PBT buffer was added to the wells. The plates were washed and bound phage was detected with anti-M13-HRP followed by TMB substrate. In these assays, phage binding to NeutrAvidin alone was tested in parallel to assess background binding. Clones whose binding signals for PCSK9 were more than 4 times higher than to NeutrAvidin (background) were considered positive. Positive clones were subjected to DNA sequence analysis.

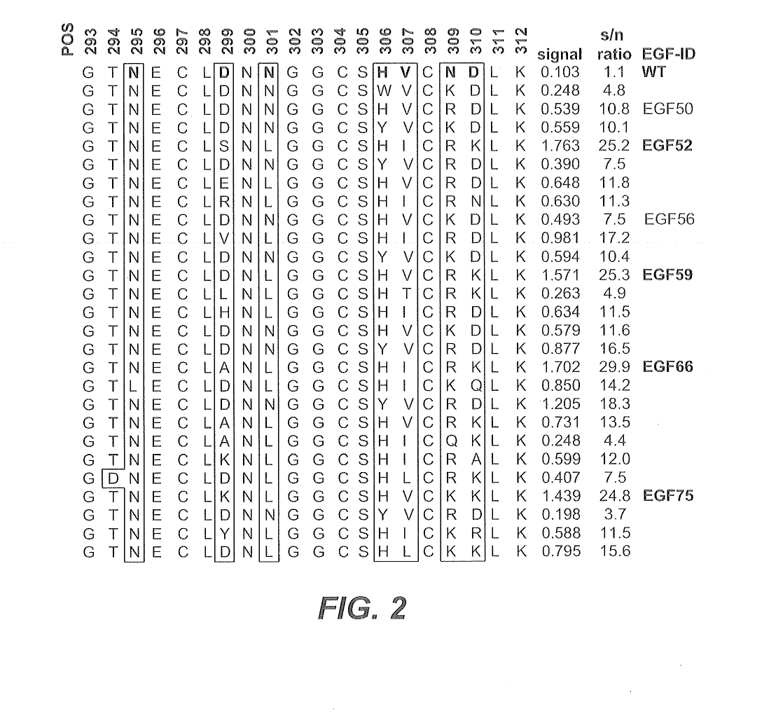

No binding signal could be detected by applying wild type EGF(A)-displaying phage to immobilized PCSK9 using a phage ELISA assay with a signal window <0.2 and a signal:noise ratio of <2 (FIG. 2, first row). After four rounds of panning, 26 unique clones were identified with moderate to strong binding signals detected by an ELISA (signal window >0.2 and signal:noise ratio >4) (FIG. 2). The sequence alignment of these clones indicated that Asn295 was highly conserved, whereas Asn309 had been mutated to either Arg or Lys. Four clones showed strong binding affinities with a signal window >1.4 and signal:noise ratio >20 and were selected for more extensive characterization. They were designated as EGF52, EGF59, EGF66 and EGF75. The major sequence variations for these four clones compared to the wild type EGF(A) (EGFwt) were Asn301 to Leu; Asn309 to Arg or Lys and Asp310 to Lys. In addition, Asp299 were changed to Ser, Ala and Lys for EGF52, EGF66 and EGF75, respectively, but remained unchanged in EGF59.

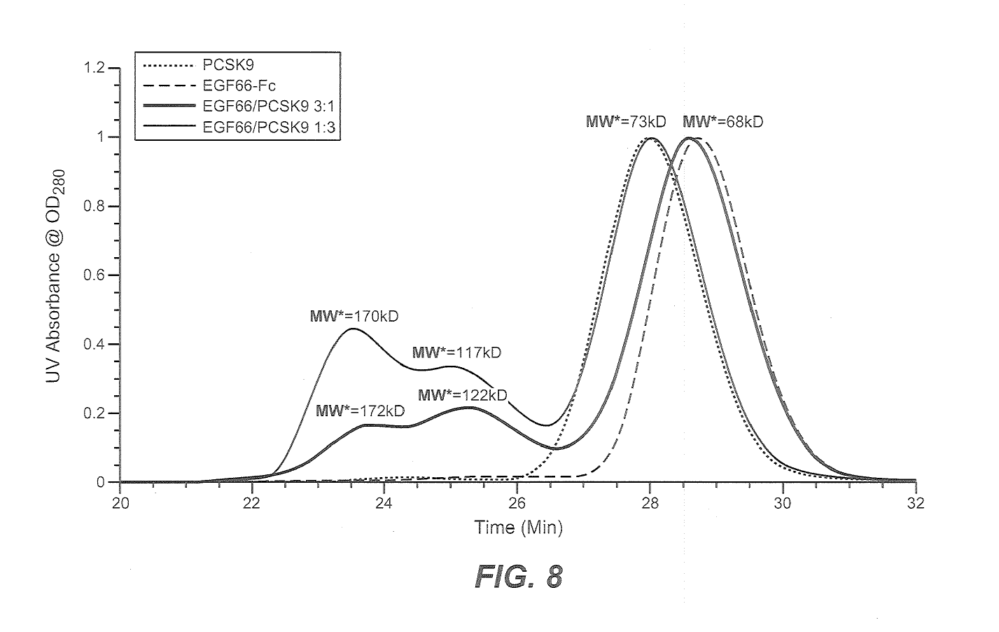

The similar spot ELISA signal for EGF52 and EGF59, which mainly differ at Asp299, suggested that this position is not critical for binding. Three clones, EGF50, EGF56 and EGF62, with single mutation at N309 to Arg, Lys and Lys, respectively, showed moderate increase of binding comparing to wild type but much lower increase compare to the four best clones. This suggests a modest contribution to binding by N309. Asn301 was mutated to Leu in all four high affinity binders, suggesting its critical role for affinity increase. To evaluate the contribution of Asp310 mutations to binding, we made a single mutation of D310K and measured the binding curve of EGF-displaying phage to PCSK9 using phage ELISA. As shown in FIG. 7, the single mutation of D310K abolished the binding completely, indicating that D310K alone could not produce an affinity increase, but has to combine with other mutations, e.g. N301L, to achieve high-affinity binding.

### Example 2

### The EGF Variants have Improved Affinities and Inhibitory Potencies

The four selected EGF variants were first made by peptide synthesis followed by in vitro folding. All EGF synthetic peptides, EGFwt, EGF52, EGF59, EGF66, and EGF75 were prepared on an automated Protein Technologies, Inc. synthesizer. Typically, the 40 amino acid peptides were assembled on Fmoc-Glu(OtBu)-Rapp polymer (substitution=0.24 meq/gm) using standard Fmoc synthesis protocols. Fmoc-Cys(Trt)-OH was incorporated for the six Cysteine amino acids. Upon completion of the linear chains, peptides were cleaved from the solid support with trifluoroacetic acid (TFA)/triisopropylsilane (TIS)/water (95:2.5:2.5) for 3 h at room temperature. TFA was evaporated and the peptides precipitated with ethyl ether, extracted with acetic acid, acetonitrile, water and lyophilized. The crude linear EGF peptides were resolubilized in DMSO and purified by reverse phase C18 chromatography using acetonitrile/water buffers. Purified fractions were analyzed by 1 cms (PE/Sciex), pooled and lyophilized.

For peptide folding, typically 50 mg of pure linear EGF peptide was dissolved in 500 ml of water (0.1 mg peptide/ml water) and the pH adjusted to >8. The linear peptides were allowed to air oxidize for 3 days at room temperature and were then lyophilized. The crude cyclic peptides were isolated by preparative reverse phase HPLC. Identity of the fully cyclized peptides were confirmed by mass spectrometry where the final masses were 6 mass units less than the linear peptides corresponding to the formation of three disulfide bonds. Cysteine pairing was as follows, Cys (I and III), Cys (II and IV), and Cys (V and VI).

The EGF variants and EGFwt were reformatted to EGF(A)-Fc fusion proteins by fusing the EGF via a short linker to the Fc domain of human IgG1. The EGF domain of LDLR (G293-E332), as well as variants described in Example 1, plus a linker with sequence of GGGSGAAQVTNKTHT (SEQ ID NO: 30) followed by Fc domain of human IgG1 (C222-K443) was cloned into pRK5 vector, designated as EGF-Fc-pRK5. The EGF-Fc protein was transiently expressed in CHO and purified on a Protein A resin followed by gel filtration chromatography. The identities of the proteins were confirmed by mass spectrometry and SDS-PAGE. Human PCSK9 (GenBank® EF692496) complementary deoxyribonucleic acids (cDNAs) containing a histidine (His)8 C-terminal tag (SEQ ID NO: 31) was cloned into a mammalian expression vector (pRK5). The recombinant human PCSK9 protein was transiently expressed in Chinese hamster ovary (CHO) cells and purified from conditioned media using affinity chromatography on a nickel nitrilotriacetic agarose column (Qiagen; Germantown, Md.) followed by gel filtration on a Sephacryl® S 200 column (GE Healthcare; Piscataway, N.J.). The identity of the protein was confirmed by mass spectrometry as well as by reducing and non reducing SDS PAGE. The protein was then biotinylated in vitro using EZ-link® Sulfo-NHS-biotinylation kit (Cat. No. 21435, Thermo Scientific, Rockford, Ill.) following the manufacturer's instruction.

Because a single EGF-Fc protein contained two EGF domains it was possible that EGF-Fc could bind to two PCSK9 simultaneously. This was examined by determining the stoichiometry of EGF66-Fc/PCSK9 complexes in solution by use of size exclusion chromatography (SEC) coupled to MALS (multi-angle light scattering). EGF66-Fc was mixed with PCSK9 in 40 mM Tris pH 7.4 with 150 mM NaCl and 2 mM CaCl₂ and incubated for 24 hours prior to analysis by size exclusion chromatography (SEC) and multi-angle light scattering (MALS). Approximately 150 μg of EGF66-Fc:PCSK9 complexes at molar ratios of 3:1, and 1:3 respectively were analyzed. Additionally, the two proteins were run independently as controls. The same buffer was used to perform separations on a Superdex 200 10/300 GL column (GE Healthcare) with a flowrate of 0.5 mL per minute. Elution profiles were monitored by UV absorbance at 280 nm (Agilent 1260 DAD), static light scatter (Wyatt Technologies Dawn Hellios-II) and differential refractive index (Wyatt Technologies Optilab rEX). The scatter intensity and the differential refractive index data were analyzed via Zimm plot with Astra 5.3.4.20 software pack (Wyatt Technologies) to determine the molar masses of the various monodispersed peaks that eluted from the Superdex 200 column.

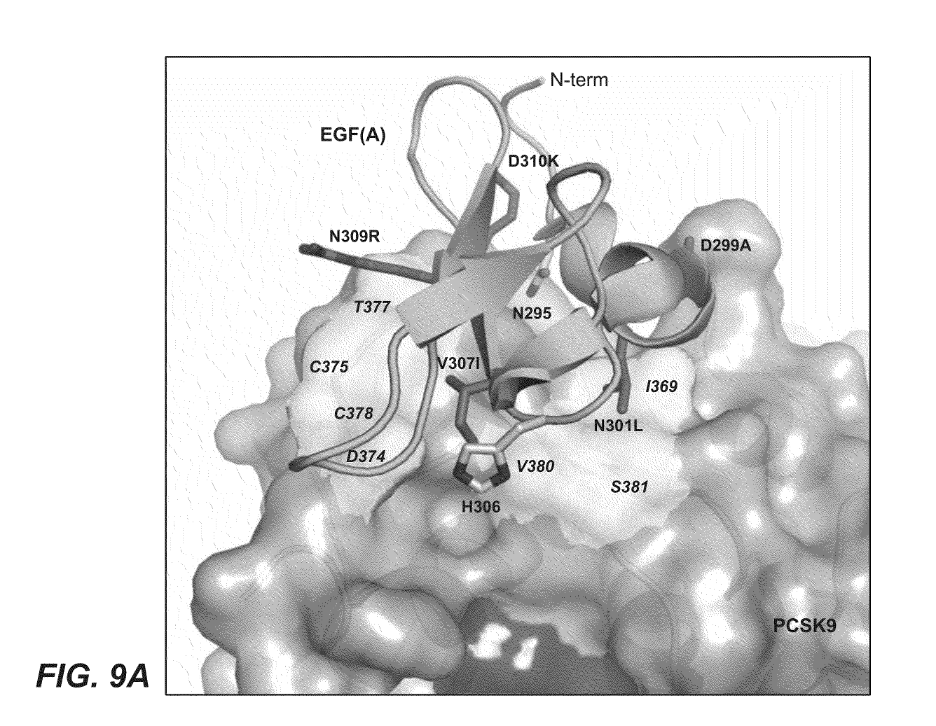

Both SEC profiles of EGF66-Fc/PCSK9 mixtures with molar ratios 1:3 or 3:1 gave two major complex peaks followed by the monomer peak of the exceeding molecule. The average molecular mass for the first peak in both cases was about 170 kDa, which is roughly consistent with stoichiometry of a 1:2 complex, and the second peak was about 120 kDa, which is consistent with a 1:1 complex (FIG. 8). In the presence of excess PCSK9 the majority of the complexes formed were 1:2 complexes, indicating that EGF-Fc proteins can bivalently interact with PCSK9.

The blocking activity of EGF peptides and EGF-Fc fusion proteins was determined by using a competition binding ELISA. Wells of 384 well MaxiSorp™ plates (Nalge Nunc International; Rochester, N.Y.) were coated overnight at 4° C. with 1 μg/mL of recombinant human LDLR extracellular domain (rLDLR) (R&D Systems; Minneapolis, Minn.) in coating buffer (50 mM sodium carbonate, pH 9.6). Then 0.5 μg/ml of biotinylated PCSK9 in assay buffer (25 mM HEPES, pH 7.2, 150 mM NaCl, 0.2 mM CaCl₂, 0.1% BSA, 0.05% Tween®20) was mixed with an equal volume of serially diluted EGF peptides (0.017-6000 nM) or EGF-Fc (0.034-6000 nM) and incubated for 30 min. The solutions were added to rLDLR coated plates and incubated for 2 h. Bound biotinylated rPCSK9 was detected by sequential additions of streptavidin-horseradish peroxidase (GE Healthcare; Buckinghamshire, UK) and substrate 3, 3′, 5, 5′ tetramethyl benzidine (TMBE 1000, Moss; Pasadena, Md.). The mean absorbance values from duplicate wells were plotted as a function of antibody concentration and the data were fitted to a four parameter equation for each antibody using KaleidaGraph (Synergy Software; Reading, Pa.).

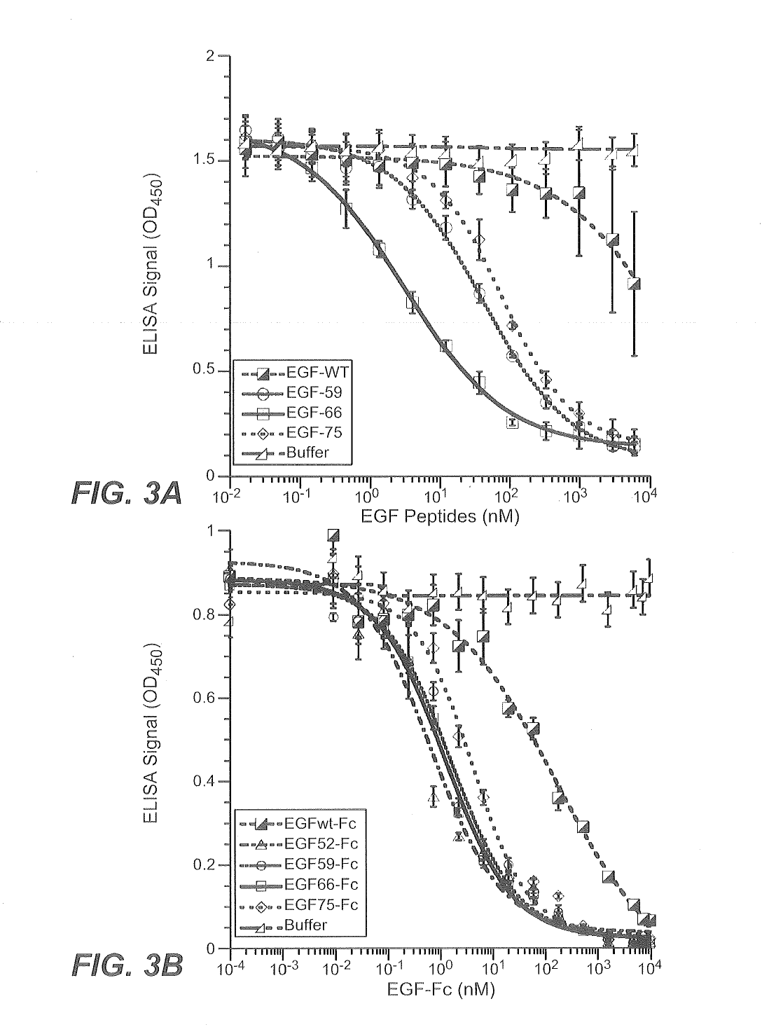

Results for the synthesized EGF peptides are shown in FIG. 3A. The IC50 values of the EGF variants were 38-247 fold lower than that of EGFwt, EGF66 being the most potent antagonist (Table I). All EGF-Fc variants displayed much better potencies in inhibiting PCSK9-LDLR binding compared to EGFwt-Fc (FIG. 3B), similar to the results with synthesized EGF peptide variants (FIG. 3A, Table I). In both assays, EGF66-Fc was the strongest antagonist.

TABLE I
Inhibition of PCSK9 binding to LDLR by EGF(A) domain variants
IC50 is the concentration at which the competitor blocked
50% of PCSK9 binding to LDLR in a competition binding
ELISA as described in Methods. Values are the average
of ±SD of three independent experiments.

| | Synthetic peptides | Fc fusion protein |
|---|---|---|
| EGF variant | IC₅₀ (nM) | IC₅₀ (nM) |
| EGFwt | >6000 | 173 ± 32 |
| EGF52 | ND | 0.7 ± 0.3 |
| EGF59 | 41.4 ± 5.1 | 1.4 ± 0.2 |
| EGF66 | 3.1 ± 0.3 | 1.1 ± 0.4 |
| EGF75 | 78.3 ± 8.5 | 4.6 ± 1.6 |

* ND, not determined

The binding affinities of the EGF-Fc fusion proteins to PCSK9 were measured by use of biolayer interferometry on an Octet RED 384 (Fortebio). Fc biosensors (Fortebio, Cat. No. 18-5063) were loaded with EGF-Fc in TrisHCl pH7.5 buffer containing 0.05% Tween20 and 0.5% BSA and 1 mM CaCl₂, washed in the same buffer and transferred to wells containing PCSK9 at concentrations ranging from 0-500 nM in the same buffer. The signal against the reference cell that contains buffer only was subtracted from all the binding data. The affinity K_{D} was obtained by non-linear fitting of the responses to a steady state algorithm using Octet software. The determined K_{D} values, summarized in Table II, show that compared to EGFwt-Fc the affinities of EGF-Fc variants increased by 7.5 to 33-fold.

TABLE II
Binding affinities of EGFwt-Fc and its variants to PCSK9
measured by biolayer interferometry. KD values were determined
by fitting the data to steady state equations. Values are
the average ± SD of three independent experiments.

| | K_{D} (steady state) (nM) |
|---|---|
| EGFwt-Fc | 900 ± 85 |
| EGF52-Fc | 120 ± 14 |
| EGF59-Fc | 50 ± 7 |
| EGF66-Fc | 56 ± 7 |
| EGF75-Fc | 27 ± 3 |

### Example 3

### Calcium-Independent Binding of EGF66-Fc to PCSK9

The interaction of the EGF(A) domain with PCSK9 requires calcium (Malby, et al. (2001) Biochemistry 40(8), 2555-2563; Saha, et al. (2001) Structure 9(6), 451-456). The side chains of residues Glu296 and Asp310 are important contributors to the coordination of a single Ca²⁺ atom by the EGF(A) domain. All EGF variants have a Lys residue at position 310 instead of the Asp310, suggesting that calcium binding is severely compromised. Therefore, we examined the calcium requirement for PCSK9 binding of EGF66-Fc in comparison with EGFwt-Fc. Binding affinities between PCSK9 and EGFwt-Fc or EGF66-Fc in the presence or absence of Ca²⁺ were determined by surface plasmon resonance on a Biacore® 3000 instrument (GE Healthcare). The sensor chip was prepared using the human antibody capture kit (Cat. No. BR-1008-39) following instructions supplied by the manufacturer. Injections of EGFwt-Fc (0.307 μg/ml) and EGF66-Fc (0.35 μg/ml), EGF75-Fc (1 μg/ml), EGF52-Fc (1 μg/ml) and EGF59-Fc (1 μg/ml) diluted in running buffer (50 mM Tris, pH 7.5, 150 mM NaCl, 0.005% P20) gave binding signals of 85.9 RU, 231.8 RU, 144 RU, 142 RU and 146 RU, respectively. Sensorgrams were recorded during a 3 min injection of PCSK9 solution in the presence of 1 mM CaCl₂ or 10 mM EDTA. Data were obtained from 2-fold serial dilutions of PCSK9 ranging from 0.078 μM to 10 μM for EGFwt-Fc and from 0 μM to 2.5 μM for EGF66-Fc with a flow rate at 30 μl/min and at a temperature of 25° C. Data were corrected by subtracting background signals of reference cells containing the capture antibody only. Kinetic parameters (ka and kd) were determined by fitting the data using Biacore® 3000 BIAevaluation software, version 4.1, and the KD values were calculated (KD=kd/ka).

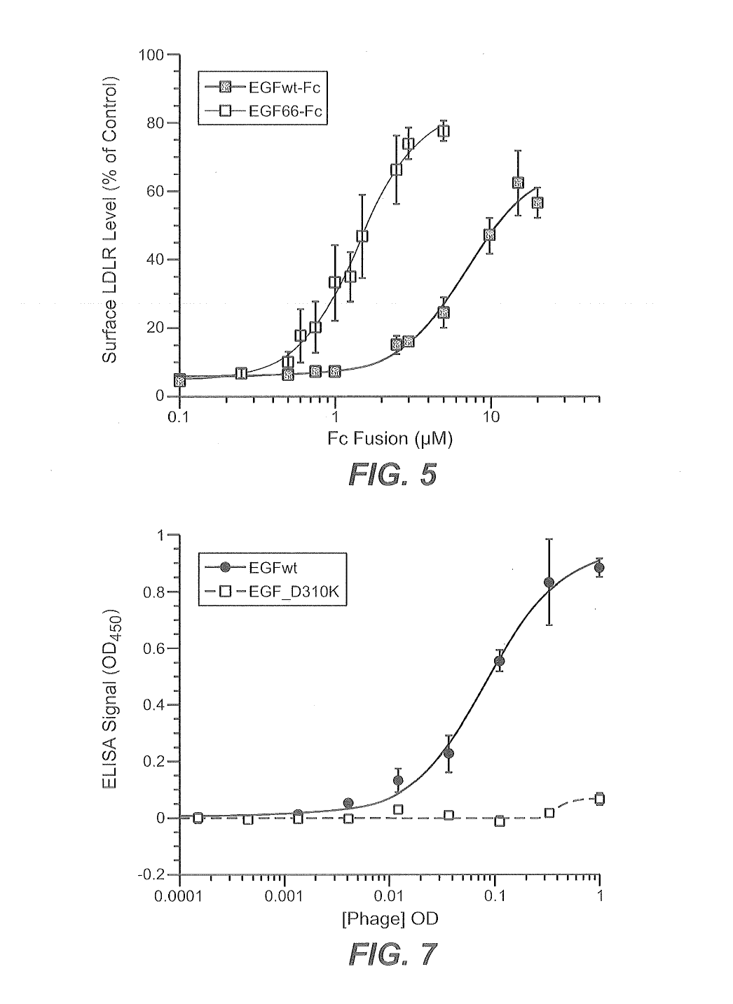

We found in these experiments that EGFwt-Fc bound to PCSK9 with a KD of 935 nM in the presence of calcium, whereas no binding signal was detected when calcium was absent (i.e., in the presence of 10 mM EDTA) (FIG. 5A, Table III). In contrast, the affinities of all four EGF mutant proteins for PCSK9 in the presence and absence of calcium were about the same (FIG. 5B, Table III). Whereas EGF66-Fc and EGF52-Fc showed virtually identical binding constants, EGF59-Fc and EGF75-Fc had only a 2-fold reduced affinity in the absence of Ca²⁺, mainly due to a 2-fold reduced kₒₙ (Table III). While not wishing to be bound by theory, the Ca²⁺-independence of the EGF variants is most likely the result of the clone selection process carried out in Ca²⁺-free buffer. This particular selection pressure favored the emergence of clones with ‘adaptive’mutations, including the Asp310 to Lysine change. In addition, the results showed that with or without Ca²⁺ present, EGF66-Fc had the highest binding affinity (K_{D} 71 nM) with an affinity improvement of about 12-fold compared to EGFwt-Fc.

TABLE III
Kinetic parameters of EGFwt-Fc or EGF52-Fc, EGF59-Fc, EGF66-Fc
or EGF75-Fc binding to PCSK9 in presence or absence Ca²⁺
measured by Surface Plasmon Resonance. Values are the
average ± SD of three independent experiments.

| | kₐ (×10⁴ M⁻¹s⁻¹) | k_{d} (×10⁻²s⁻¹) | K_{D} (nM) |
|---|---|---|---|
| EGFwt-Fc, 1 mM Ca²⁺ | 5.9 ± 0.4 | 5.5 ± 0.5 | 935 ± 6 |
| EGFwt-Fc, 10 mM EDTA | ND* | ND | ND |
| EGF52-Fc, 1 mM Ca²⁺ | 18.1 ± 0.7 | 2.0 ± 0.9 | 113 ± 9 |
| EGF52-Fc, 10 mM EDTA | 9.0 ± 0.1 | 2.2 ± 0.2 | 238 ± 8 |
| EGF59-Fc, 1 mM Ca²⁺ | 18.3 ± 0.3 | 2.0 ± 0.3 | 111 ± 4 |
| EGF59-Fc, 10 mM EDTA | ±15.6 ± 0.7 | 2.1 ± 0.6 | 135 ± 4 |
| EGF66-Fc, 1 mM Ca²⁺ | 32.6 ± 2.5 | 2.3 ± 0.1 | 71 ± 1 |
| EGF66-Fc, 10 mM EDTA | 32.4 ± 1.1 | 2.3 ± 0.2 | 72 ± 2 |
| EGF75-Fc, 1 mM Ca²⁺ | 17 ± 0.2 | 2.0 ± 0.6 | 121 ± 9 |
| EGF75-Fc, 10 mM EDTA | 9.5 ± 0.2 | 2.1 ± 0.8 | 224 ± 3 |

*ND not detected

### Example 4

### In Vitro and In Vivo Efficacy of EGF66-Fc

Based on its superior inhibitory activity, EGF66-Fc was used as a PCSK9 antagonist in an LDLR degradation assay with HepG2 cells. HepG2 cells (ATCC; Manassas, Va.) were seeded into 48 well plates (Corning; Corning, N.Y.) at 1×10⁵ cells per well in high glucose medium (DMEM, Gibco; Carlsbad, Calif.) containing 2 mM glutamine (Sigma), penicillin/streptomycin (Gibco) and 10% FBS (Sigma) and incubated overnight. Then the medium was changed to DMEM containing 10% lipoprotein deficient serum (LPDS, Intracel; Frederick, Md.). After 24 h, 15 μg/ml PCSK9 was mixed with serially diluted EGFwt-Fc and EGF66-Fc fusion proteins, added to the cells and incubated at 37° C. for 4 h. Cells were rinsed with PBS and detached using 2.5 mM EDTA (EMD; Gibbstown, N.J.). After centrifugation, the resuspended cells were incubated with 1:20 anti-LDLR antibody (Progen Biotechnik; Heidelberg, Germany) on ice for 15 min. The samples were then washed with PBS and incubated with 1:200 diluted goat anti mouse IgG Alexa Fluor® 488 (Invitrogen; Carlsbad, Calif.) on ice for 15 min. After two PBS washes cells were resuspended in PBS containing 10 μg/ml of propidium iodide and analyzed on a dual laser flow cytometer (FACScan, Becton Dickinson; Franklin Lakes, N.J.). Relative fluorescence units (RFUs) were used to quantify LDLR expression levels on the HepG2 cell surface. Cell surface LDLR levels were expressed as percent of LDLR levels measured in the absence of PCSK9 (=control).

EGF66-Fc protein prevented PCSK9-mediated LDLR degradation in a concentration-dependent manner (FIG. 5). At the highest concentration tested (5 μM) the LDLR surface levels were about 80% of control levels measured in the absence of PCSK9. In comparison, the EGFwt-Fc was much less potent in restoring LDLR surface levels (FIG. 5) reaching 56% of control levels at the highest concentration tested (20 μM). The concentrations that restored LDLR levels to 50% of control (effective concentration, EC₅₀) were 1.6 μM and 11 μM for EGF66-Fc and EGFwt-Fc, respectively.

To determine whether increased affinity and cell efficacy could translate into improved therapeutic potential, we compared the effects of EGFwt-Fc and EGF66-Fc in rescuing liver LDLR upon treatment with PCSK9 in a mouse model. Eight weeks old male C57BL/6 mice were purchased from approved vendor and housed for 2 weeks before starting the experiment. Mice were randomized into 3 groups (3 mice/group) based on body weight and given either EGFwt-Fc or EGF66-Fc fusion proteins or PBS (vehicle/control) at the indicated dose through the i.v. route. After 2 h, mice were dosed i.v. with 30 μg of PCSK9 in PBS. After 1 h livers were harvested and snap frozen.

Approximately 200 mg of each liver were homogenized in Extraction Buffer 1 supplemented with Protease Inhibitor Cocktail (ProteoExtract® Native Membrane Protein Extraction Kit, Cat. No. 444810, Calbiochem) using the TissueLyser (Qiagen) according to manufacturer's instructions. Lysates were centrifuged and the cell pellet was resuspended in Extraction Buffer II supplemented with Protease Inhibitor Cocktail (Calbiochem). After 30 min of gentle agitation at 4° C., the samples were centrifuged and the supernatants containing the membrane proteins were quantified using the Bradford assay. 4×SDS sample buffer was added. For each group (n=3), liver proteins were pooled for a total of 100 μg of protein and boiled for 5 min. The samples were loaded onto a 4-12% Bis-Tris Midi gel and proteins separated by SDS-PAGE. After transfer to nitrocellulose membranes using the iBlot® (Invitrogen), membranes were blocked with 5% nonfat milk for 1 h at room temperature. The blots were incubated with 1:200 anti-LDLR (Abcam) in 5% nonfat milk overnight at 4° C. Blots were washed three times with TBS-T (10 mM TRIS, pH 8.0, 150 mM NaCl, 0.1% Tween®20) for 15 min. Blots were then incubated with 1:5000 anti-rabbit horseradish peroxidase (GE Healthcare) in 5% nonfat milk for 1 hour. After washing with TBS-T, proteins were visualized using ECL-Plus (GE Healthcare) and exposure to XAR film (Kodak). The membranes were then washed with TBS-T and incubated with 1:5000 anti-transferrin receptor (Invitrogen) for 2 hours at room temperature. After washing with TBS-T, the membrane was incubated in 1:10000 anti-mouse horseradish peroxidase (GE Healthcare) for 1 hour and washed again. Proteins were visualized using ECL Plus and exposure to XAR film.

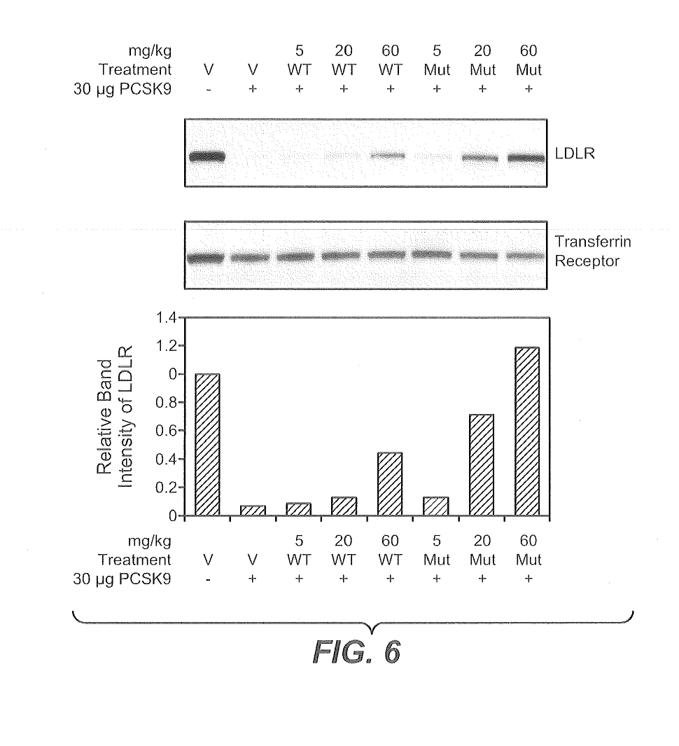

Mice were first injected with vehicle, EGFwt-Fc and EGF66-Fc followed by a bolus of recombinant human PCSK9 (30 μg/mouse) and livers were collected and analyzed 1 h later. As shown in FIG. 6, treatment of PCSK9 dramatically reduced liver LDLR to <10% of normal levels (without PCSK9 treatment). Pre-treatment with EGFwt-Fc rescued liver LDLR to less than 50% of control levels at the highest dose (60 mg/kg), whereas pre-treating with EGF66-Fc could rescue LDLR level to 70% at the medium dose of 20 mg/kg and to ˜100% at the highest dose (60 mg/kg). The results suggested that the improved affinity of EGF66 translated into a significantly improved antagonistic potency in vivo.

### Example 5

### Structural Analysis of EGF Variants

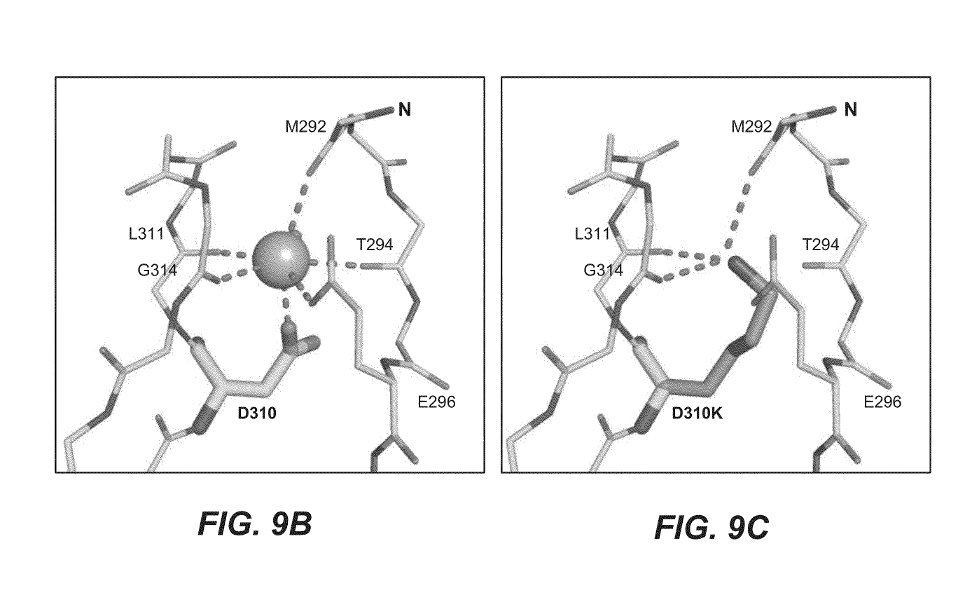

A model of EGF66 was generated to investigate why EGF66 binding to PCSK9 did not require calcium and why particular amino acids were selected during the phage optimization process (FIG. 9A). The mutations present in EGF66 were manually modeled with PyMOL (The PyMOL Molecular Graphics System, V1.2r3pre, Schrödinger LLC) using the structure of the complex between PCSK9 and the EGF(A) domain of the LDL receptor (PDB Accession code 3BPS) (Kwon, et al., supra). In all five cases, the mutation could be accommodated without the need for any changes in backbone conformation. Side chain geometries from the standard PyMOL rotamer libraries were selected so as to minimize clashes with other atoms of the EGF domain or atoms of PCSK9. The geometries in this library are derived from commonly occurring side chain conformations in published protein structures and therefore represent low energy states. In the case of D310K, the initial low energy lysine side chain conformation was augmented with a ˜10° shift in chi-3 and a ˜120° change in chi-4 so as to bring the N^{ε} atom in the vicinity of the Ca²⁺ ion observed in the wild-type protein. Since all of the side chain dihedral angles are staggered and there are minimal clashes with other protein atoms, a lysine at this position can adopt low energy conformations with the ammonium ion in the Ca²⁺-binding loop without significant changes in backbone conformation.

D299 is preserved in 14 of the 26 phage sequences. Although slightly farther than hydrogen bonding distance from the N-terminus of PCSK9 (S 153), the aspartate side chain may be involved in favorable polar contact with the PCSK9 N-terminal amine (Bottomley, et al., supra). The reason for selection of alanine at this position in EGF66 is not readily apparent from the modeled structure. N301 in wild-type EGF is involved in two intramolecular hydrogen bonds but does not make any intermolecular contacts to PCSK9. The wild-type residue is maintained (10 cases) or replaced by leucine (16 cases) during the phage selection. The model of EGF66 suggests that leucine in this position could participate in favorable hydrophobic interactions with 1369 (Cγ1 and Cδ1), V380 (Cα) and S381 (Cβ) of PCSK9. V307 is located at one end of the main EGF β-hairpin. The majority of phage selections at this site are β-branched amino acids that would all help to stabilize the β-strand conformation. Moreover, the V3071 replacement in EGF66 might also permit additional hydrophobic contacts with D374 (Cβ), V380 (Cγ2) or C378 (Sγ) of PCSK9. The side chain of N309 is involved in two hydrogen bonds, one intramolecular (to E316 Oε) and one intermolecular (to PCSK9-T377Oγ1). All but one of the phage clones replaced N309 with a basic residue. This preference may be driven by increased interactions with E316 (stabilizing the EGF β-hairpin) or by improved hydrophobic contacts between the methylene groups of a basic residue and a non-polar patch on the PCSK9 surface formed by the C375-C378 disulfide and the methyl group of T377.

Two additional residues were varied in the phage-libraries but maintained the wild-type residue in EGF66. Asparagine at residue 295 is present in all but one of the phage sequences, suggesting the importance of its two side chain hydrogen bond interactions (intramolecular to C297 backbone N and intermolecular to D23806). Residue 306 is a histidine in wild-type EGF domain and has been proposed to contribute to the increased affinity of LDLR for PCSK9 at low pH via a charge-charge interaction with D374 of PCSK9 (Bottomley, et al., supra). The imidazole ring also packs against the side chain of P320 within the EGF domain. The aromatic character of H306 is preserved in all of the phage sequences (His, Trp, Tyr). Histidine, tryptophan and tyrosine would all be able to contact the P320 side chain, suggesting that this ring stacking may be important for stabilizing the orientation of the N- and C-terminal subdomains of EGF. EGF-H306Y has previously been shown to bind more tightly to PCSK9, rationalized by the potential formation of a direct hydrogen bond to D374 (Bottomley, et al., supra).

Chelation of Ca²⁺ is a common feature of EGF domains, and is hypothesized to stabilize the domain fold and also speculated to play a role in inter-domain interactions (Handford et al. (1991) Nature 351: 164-167). The EGF(A) domain of LDLR chelates Ca²⁺, and the side chain of D310 plays a key role in contacting the ion. Moreover, binding of EGF to PCSK9 is Ca²⁺-dependent. Given this role, it is perhaps not surprising that 13 of the 26 phage-derived sequences preserve the aspartate at this site. However, 9 of the 26 phage-derived sequences have D310 replaced by lysine, which would be incapable of chelating Ca⁺². Of note, the phage selection was performed in the absence of exogenously added Ca⁺², which may have added selection pressure for phage clones with compensatory amino acid changes at this position. While not wishing to be bound by theory, the D310K mutation may relieve the need for Ca⁺² to render EGF competent for PCSK9 binding. One interesting possibility is that the side chain amino group of K310 plays a similar role to the Ca²⁺ ion by using polar interactions to bridge between the 309-316 β-hairpin (backbone oxygen of L311 and G314) and the N-terminal strand of EGF66 (e.g. backbone oxygen of M292 and T294; side chain of E296) thereby stabilizing packing of the latter onto the EGF domain (FIG. 9B).

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A PCSK9-binding polypeptide comprising the amino acid sequence:
GX₁X₂ECLX₃NX₄GGCSX₅X₆CX₇X₈LKIGYECLCPDGFQLVAQRRCE, wherein X₁ is D or T; X₂ is L or N; X₃ is selected from the group consisting of A, D, E, H, K, L, R, S, V, and Y; X₄ is L or N; X₅ is selected from the group consisting of H, W, and Y; X₆ is selected from the group consisting of I, L, T and V; X₇ is selected from the group consisting of K, N, R and Q; and X₈ is selected from the group consisting of A, D, K, N, Q and R (SEQ ID NO: 1).

2. The polypeptide of claim 1, wherein said polypeptide comprises an amino acid sequence selected from the group consisting of SEQ ID NOs: 2-27.

3. The polypeptide of claim 1 further comprising an immunoglobulin sequence.

4. The polypeptide of claim 3, wherein said immunoglobulin sequence is an antibody constant region.

5. The polypeptide of claim 4, wherein said antibody constant region is an Fc region.

6. The polypeptide of claim 5, wherein said Fc region is from an IgG antibody.

7. An isolated nucleic acid encoding the polypeptide of claim 1.

8. A vector comprising the nucleic acid of claim 7.

9. The vector of claim 8, wherein said vector is an expression vector.

10. A host cell comprising the vector of claim 8.

11. The host cell of claim 10, wherein the host cell is prokaryotic.

12. The host cell of claim 10, wherein the host cell is eukaryotic.

13. A method for making the polypeptide of claim 1, said method comprising culturing the host cell of claim 10 under conditions suitable for expression of the nucleic acid encoding said polypeptide.

14. The method of claim 13, further comprising recovering the polypeptide from the host cell.

15. A pharmaceutical composition comprising the polypeptide of claim 1 and a pharmaceutically acceptable carrier.

16. A method of reducing LDL-cholesterol level in a subject, said method comprising administering to the subject an effective amount of the polypeptide of claim 1.

17. A method of treating cholesterol related disorder in a subject, said method comprising administering to the subject an effective amount of the polypeptide of claim 1.

18. A method of treating hypercholesterolemia in a subject, said method comprising administering to the subject an effective amount of the polypeptide of claim 1.

19. The method of claim 16, 17 or 18, further comprising administering to the subject an effective amount of a second medicament, wherein the polypeptide is the first medicament.

20. The method of claim 19, wherein the second medicament elevates the level of LDLR.

21. The method of claim 19, wherein the second medicament reduces the level of LDL-cholesterol.

22. The method of claim 19, wherein the second medicament comprises a statin.

23. The method of claim 22, wherein the statin is selected from the group consisting of atorvastatin, fluvastatin, lovastatin, mevastatin, pitavastatin, pravastatin, rosuvastatin, simvastatin, and any combination thereof.

24. The method of claim 19, wherein the second medicament elevates the level of HDL-cholesterol.

25. A method of inhibiting binding of PCSK9 to LDLR in a sample, the method comprising adding the polypeptide of claim 1 to the sample.

26. A method of inhibiting binding of PCSK9 to LDLR in a subject, said method comprising administering to the subject an effective amount of the polypeptide of claim 1.

27. A method of detecting PCSK9 protein in a sample, said method comprising
(a) contacting the sample with the polypeptide of claim 1; and
(b) detecting formation of a complex between the polypeptide and the PCSK9 protein.


SEQUENCE LISTING (31 sequences)

SEQ ID NO: 1 - protein, 40 aa, Artificial Sequence
  Description of Artificial Sequence Synthetic polypeptide
GXXECLXNXGGCSXXCXXLKIGYECLCPDGFQLVAQRRCE

SEQ ID NO: 2 - protein, 20 aa, Artificial Sequence
  Description of Artificial Sequence Synthetic peptide
GTNECLDNNGGCSWVCKDLK

SEQ ID NO: 3 - protein, 20 aa, Artificial Sequence
  Description of Artificial Sequence Synthetic peptide
GTNECLDNNGGCSHVCRDLK

SEQ ID NO: 4 - protein, 20 aa, Artificial Sequence
  Description of Artificial Sequence Synthetic peptide
GTNECLDNNGGCSYVCKDLK

SEQ ID NO: 5 - protein, 20 aa, Artificial Sequence
  Description of Artificial Sequence Synthetic peptide
GTNECLSNLGGCSHICRKLK

SEQ ID NO: 6 - protein, 20 aa, Artificial Sequence
  Description of Artificial Sequence Synthetic peptide
GTNECLDNNGGCSYVCRDLK

SEQ ID NO: 7 - protein, 20 aa, Artificial Sequence
  Description of Artificial Sequence Synthetic peptide
GTNECLENLGGCSHVCRDLK

SEQ ID NO: 8 - protein, 20 aa, Artificial Sequence
  Description of Artificial Sequence Synthetic peptide
GTNECLRNLGGCSHICRNLK

SEQ ID NO: 9 - protein, 20 aa, Artificial Sequence
  Description of Artificial Sequence Synthetic peptide
GTNECLDNNGGCSHVCKDLK

SEQ ID NO: 10 - protein, 20 aa, Artificial Sequence
  Description of Artificial Sequence Synthetic peptide
GTNECLVNLGGCSHICRDLK

SEQ ID NO: 11 - protein, 20 aa, Artificial Sequence
  Description of Artificial Sequence Synthetic peptide
GTNECLDNNGGCSYVCKDLK

SEQ ID NO: 12 - protein, 20 aa, Artificial Sequence
  Description of Artificial Sequence Synthetic peptide
GTNECLDNLGGCSHVCRKLK

SEQ ID NO: 13 - protein, 20 aa, Artificial Sequence
  Description of Artificial Sequence Synthetic peptide
GTNECLLNLGGCSHTCRKLK

SEQ ID NO: 14 - protein, 20 aa, Artificial Sequence
  Description of Artificial Sequence Synthetic peptide
GTNECLHNLGGCSHICRDLK

SEQ ID NO: 15 - protein, 20 aa, Artificial Sequence
  Description of Artificial Sequence Synthetic peptide
GTNECLDNNGGCSHVCKDLK

SEQ ID NO: 16 - protein, 20 aa, Artificial Sequence
  Description of Artificial Sequence Synthetic peptide
GTNECLDNNGGCSYVCRDLK

SEQ ID NO: 17 - protein, 20 aa, Artificial Sequence
  Description of Artificial Sequence Synthetic peptide
GTNECLANLGGCSHICRKLK

SEQ ID NO: 18 - protein, 20 aa, Artificial Sequence
  Description of Artificial Sequence Synthetic peptide
GTLECLDNLGGCSHICKQLK

SEQ ID NO: 19 - protein, 20 aa, Artificial Sequence
  Description of Artificial Sequence Synthetic peptide
GTNECLDNNGGCSYVCRDLK

SEQ ID NO: 20 - protein, 20 aa, Artificial Sequence
  Description of Artificial Sequence Synthetic peptide
GTNECLANLGGCSHVCRKLK

SEQ ID NO: 21 - protein, 20 aa, Artificial Sequence
  Description of Artificial Sequence Synthetic peptide
GTNECLANLGGCSHICQKLK

SEQ ID NO: 22 - protein, 20 aa, Artificial Sequence
  Description of Artificial Sequence Synthetic peptide
GTNECLKNLGGCSHICRALK

SEQ ID NO: 23 - protein, 20 aa, Artificial Sequence
  Description of Artificial Sequence Synthetic peptide
GDNECLDNLGGCSHLCRKLK

SEQ ID NO: 24 - protein, 20 aa, Artificial Sequence
  Description of Artificial Sequence Synthetic peptide
GTNECLKNLGGCSHVCKKLK

SEQ ID NO: 25 - protein, 20 aa, Artificial Sequence
  Description of Artificial Sequence Synthetic peptide
GTNECLDNNGGCSYVCRDLK

SEQ ID NO: 26 - protein, 20 aa, Artificial Sequence
  Description of Artificial Sequence Synthetic peptide
GTNECLYNLGGCSHICKRLK

SEQ ID NO: 27 - protein, 20 aa, Artificial Sequence
  Description of Artificial Sequence Synthetic peptide
GTNECLDNLGGCSHLCKKLK

SEQ ID NO: 28 - protein, 20 aa, Homo sapiens
GTNECLDNNGGCSHVCNDLK

SEQ ID NO: 29 - protein, 20 aa, Homo sapiens
IGYECLCPDGFQLVAQRRCE

SEQ ID NO: 30 - protein, 15 aa, Artificial Sequence
  Description of Artificial Sequence Synthetic peptide
GGGSGAAQVTNKTHT

SEQ ID NO: 31 - protein, 8 aa, Artificial Sequence
  Description of Artificial Sequence Synthetic 8xHis tag
HHHHHHHH